(19) United States
(12) Patent Application Publication
(10) Pub. No.: US 2015/0196276 A1
(43) Pub. Date: Jul. 16, 2015

(54) ULTRASONIC PROBE AND METHOD OF MANUFACTURING THE SAME

(71) Applicant: SAMSUNG MEDISON CO., LTD., Hongcheon-gun (KR)

(72) Inventors: Min-seon SEO, Gyeongju-si (KR); Jae-yk KIM, Seongnam-si (KR)

(21) Appl. No.: 14/597,123

(22) Filed: Jan. 14, 2015

(30) Foreign Application Priority Data
Jan. 14, 2014 (KR) 10-2014-0004708
Jul. 29, 2014 (KR) 10-2014-0096761

(51) Int. Cl. A61B 8/00; A61B 8/14

(52) U.S. Cl. CPC A61B 8/4494; A61B 8/14; A61B 8/4444

(57) ABSTRACT

Provided are an ultrasonic probe and a method of manufacturing the same. The ultrasonic probe includes a body in which grooves are formed, and ultrasonic transducers disposed respectively in the grooves to perform transduction between an ultrasonic signal and an electrical signal.

### RELATED APPLICATIONS

This application claims the benefit of Korean Patent Application No. 10-2014-0004708, filed on Jan. 14, 2014 and NO 10-2014-0096761, filed on Jul. 29, 2014 in the Korean Intellectual Property Office, the disclosure of which is incorporated herein in its entirety by reference.

### BACKGROUND

1. Field

One or more embodiments of the present invention relate to ultrasonic probes and methods of manufacturing the same.

2. Description of the Related Art

In general, an ultrasonic diagnosis apparatus irradiates ultrasound onto an object of living organisms such as a human or an animal, detects an echo signal reflected from the object, displays a tomographic image of tissue of the living organism on a monitor, and provides information necessary for a diagnosis of the object.

The ultrasonic diagnosis apparatus includes an ultrasonic probe for transmitting an ultrasound into the object and receiving an echo signal from the object. The ultrasonic probe includes an ultrasonic transducer that is installed in the ultrasonic probe and performs transduction between an ultrasonic signal and an electrical signal. The ultrasonic transducer may noninvasively obtain a picture or image of tissue or an organ of a body.

A typical example of the ultrasonic transducer is a piezoelectric micromachined ultrasonic transducer (PMUT) that vibrates according to pressure and performs transduction between an ultrasonic signal and an electrical signal. However, a capacitive micromachined ultrasonic transducer (CMUT) that vibrates according to electrostatic capacity and performs transduction between an ultrasonic signal and an electrical signal has recently been developed.

The CMUT may transmit a wideband-frequency ultrasonic signal, but a manufacturing method thereof is complex and crosstalk occurs between CMUTs.

### SUMMARY

One or more embodiments of the present invention include ultrasonic probes capable of reducing crosstalk between ultrasonic transducers, and methods of manufacturing the same.

One or more embodiments of the present invention include ultrasonic probes having a large ultrasonic transmission angle, and methods of manufacturing the same.

Additional aspects will be set forth in part in the description which follows and, in part, will be apparent from the description, or may be learned by practice of the presented embodiments.

According to one or more embodiments of the present invention, an ultrasonic probe includes: a body in which a plurality of grooves are formed; and a plurality of ultrasonic transducers disposed respectively in the grooves to perform transduction between an ultrasonic signal and an electrical signal.

The body may include a sound-absorbing material.

The ultrasonic transducers may be disposed on bottom surfaces of the grooves.

The bottom surfaces may be flat.

The ultrasonic transducers may transmit ultrasonic signals in directions perpendicular to the bottom surfaces of the grooves.

The grooves may include a first groove and a second groove that are spaced apart from each other, and the ultrasonic transducers may include a first ultrasonic transducer and a second ultrasonic transducer that are disposed respectively in the first groove and the second groove.

A spacing portion, which is a portion of the body, may be disposed between the first groove and the second groove.

A frequency of an ultrasonic signal transmitted by the first ultrasonic transducer may be different from a frequency of an ultrasonic signal transmitted by the second ultrasonic transducer.

The body may include a curved surface, and the grooves may be formed in the curved surface.

The curved surface may be convexed outward.

The ultrasonic probe may have an ultrasonic transmission angle of about 120° to about 360°.

The grooves may be arranged one-dimensionally or two-dimensionally.

The ultrasonic transducers may be capacitive micromachined ultrasonic transducers (CMUTs).

The ultrasonic transducers may include at least one ultrasonic cell including a vibration portion vibrating according to an applied voltage, a first electrode contacting the vibration portion, and a second electrode contacting the vibration portion and spaced apart from the first electrode.

The ultrasonic transducers may include a first ultrasonic cell vibrating in a first frequency band and a second ultrasonic cell vibrating in a second frequency band.

According to one or more embodiments of the present invention, a method of manufacturing an ultrasonic probe includes: forming grooves in a sound-absorbing material of a body; and disposing ultrasonic transducers, which perform transduction between an ultrasonic signal and an electrical signal, respectively in the grooves.

A spacing portion, which is a portion of the body, may be formed between the grooves.

The grooves may be formed on a curved surface of the body.

The grooves may have flat bottom surfaces.

The ultrasonic probe may operate at multiband frequencies.

Crosstalk between the ultrasonic transducers may be reduced.

The ultrasonic probe and the method of manufacturing the same may improve durability.

### BRIEF DESCRIPTION OF THE DRAWINGS

These and/or other aspects will become apparent and more readily appreciated from the following description of the embodiments, taken in conjunction with the accompanying drawings in which:

FIG. 1 is a block diagram of an ultrasonic diagnosis apparatus according to an embodiment of the present invention;

FIG. 2 is a block diagram of an ultrasonic probe illustrated in FIG. 2;

FIG. 3 is a plan view of an ultrasonic transducer illustrated in FIG. 2;

FIG. 4 is a schematic cross-sectional view of an ultrasonic cell illustrated in FIG. 3;

FIGS. 5A to 5F are cross-sectional views illustrating various examples of an electrode arrangement of an ultrasonic cell;

FIG. 6 is a schematic cross-sectional view of a body in which ultrasonic transducers are arranged according to an embodiment of the present invention;

FIG. 7 is a flowchart of a method of manufacturing an ultrasonic probe, according to an embodiment of the present invention;

FIGS. 8A to 8C illustrate ultrasonic transducers according to embodiments of the present invention;

FIGS. 9A and 9B illustrate an arrangement relation between ultrasonic cells operating at different frequencies, according to an embodiment of the present invention; and

FIGS. 10A and 10B illustrate an arrangement relation between ultrasonic transducers operating at different frequencies, according to an embodiment of the present invention.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, expressions such as “at least one of,” when preceding a list of elements, modify the entire list of elements and do not modify the individual elements of the list.

Hereinafter, embodiments of the present invention will be described in detail with reference to the accompanying drawings. In the following description, like reference numerals denote like elements, and redundant descriptions thereof will be omitted.

Throughout the specification, an “object” may include a human, an animal, or a part of a human or animal. For example, the object may include organs such as a liver, heart, womb, brain, breast, abdomen, or the like, or a blood vessel.

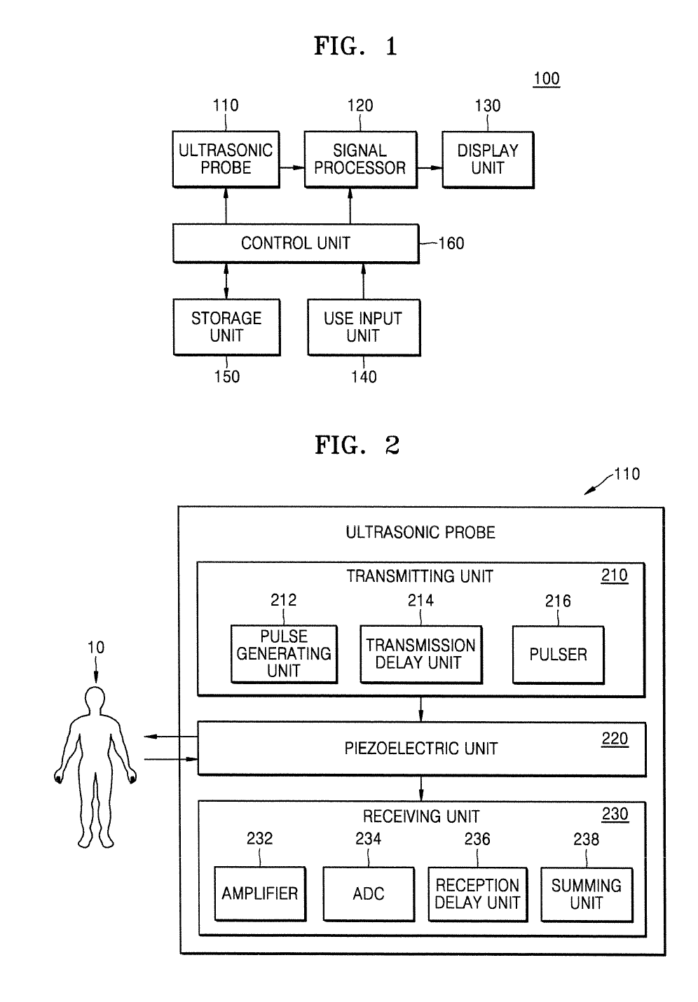

FIG. 1 is a block diagram of an ultrasonic diagnosis apparatus 100 according to an embodiment of the present invention. Referring to FIG. 1, the ultrasonic diagnosis apparatus 100 includes an ultrasonic probe 110 that transmits/receives an ultrasonic signal, a signal processing unit 120 that processes a signal applied to the ultrasonic probe 110 and generates an image, a display unit 130 that displays an image, a user input unit 140 that receives an input of a user command, a storage unit 150 that stores various information, and a control unit 160 that controls overall operations of the ultrasonic diagnosis apparatus 100.

The ultrasonic probe 110 transmits an ultrasonic signal to an object and receives an echo signal reflected from the object, as will be described later in detail.

The signal processing unit 120 processes ultrasonic data generated by the ultrasonic probe 110 and generates an ultrasonic image. The ultrasonic image may be at least one of a brightness (B) mode image representing a magnitude of an ultrasonic echo signal by brightness, a Doppler mode image representing an image of a moving object in the form of a spectrum by using the Doppler effect, a motion (M) mode image representing a time-dependent motion of an object at a predetermined position, an elasticity mode image representing a difference between a reaction of an object when compression is applied to the object and a reaction of the object when compression is not applied to the object by an image, and a color (C) mode image representing a speed of a moving object by color by using the Doppler effect. The ultrasonic image is generated by a known ultrasonic image generating method, and thus a detailed description thereof will be omitted. Accordingly, the ultrasonic image according to an embodiment of the present invention may include 1D, 2D, 3D, and 4D mode images.

The display unit 130 displays information processed by the ultrasonic diagnosis apparatus 100. For example, the display unit 130 may display an ultrasonic image generated by the signal processing unit 120 and may display a graphical user interface (GUI) via which a user input is received.

The display unit 130 may include at least one of a liquid crystal display, a thin film transistor liquid crystal display, an organic light-emitting diode display, a flexible display, a three-dimensional (3D) display, and an electrophoretic display, and the ultrasonic diagnosis apparatus 100 may include two or more display units 130 according to embodiments.

The user input unit 140 refers to a unit through which a user inputs data for controlling the ultrasonic diagnosis apparatus 100. The user input unit 140 may include a keypad, a mouse, a touch panel, and a track ball. However, the user input unit 140 is not limited thereto, and may further include other input units such as a jog wheel and a jog switch.

The touch panel may detect a real touch of a pointer to a screen and may also detect a proximity touch of a pointer to a screen when the pointer approaches within a predetermined distance from the screen. In this specification, the pointer refers to a tool for performing a proximity touch or a touch to a predetermined portion of the touch panel, examples of which may include a stylus pen or a body part such as a finger.

The touch panel may include a touchscreen forming a layer structure with the display unit 130, and the touchscreen may be any one of various types such as a capacitive overlay type, a resistive overlay type, an infrared beam type, an integral strain gauge type, a surface acoustic wave type, and a piezoelectric type. The touchscreen may function as not only the display unit 130 but also the user input unit 140.

Although not illustrated, various sensors may be provided in or near the touch panel to sense a touch to the touch panel. An example of the sensor for sensing a touch to the touch panel is a tactile sensor. The tactile sensor refers to a sensor that senses a touch of an object at the degree of human sense or more. The tactile sensor may sense a variety of information, such as the roughness of a touch surface, the hardness of a touch object, and the temperature of a touch point.

Another example of the sensor for sensing a touch to the touch panel is a proximity sensor. The proximity sensor refers to a sensor that detects the presence of an object approaching a predetermined detection surface or an object located in the proximity thereof without mechanical contact by using an electromagnetic force or infrared rays. Examples of the proximity sensor may include transmission type photoelectric sensors, direct reflection type photoelectric sensors, mirror reflection type photoelectric sensors, high frequency oscillation type proximity sensors, electrostatic capacity type proximity sensors, magnetic type proximity sensors, and infrared proximity sensors.

The storage unit 150 stores various information processed by the ultrasonic diagnosis apparatus 100. For example, the storage unit 150 may store medical data (e.g., images) related to a diagnosis of an object and may also store algorithms or programs to be executed in the ultrasonic diagnosis apparatus 100.

The storage unit 150 may include at least one storage medium from among a flash memory-type storage medium, a hard disk-type storage medium, a multimedia card micro-type storage medium, card-type memories (e.g., an SD card, an XD memory, and the like), Random Access Memory (RAM), Static Random Access Memory (SRAM), Read-Only Memory (ROM), Electrically Erasable Programmable Read-Only Memory (EEPROM), Programmable Read-Only Memory (PROM), a magnetic memory, a magnetic disk, and an optical disk. Also, the ultrasonic diagnosis apparatus 100 may utilize a web storage or a cloud server that functions as the storage unit 150 online.

The control unit 160 may control overall operations of the ultrasonic diagnosis apparatus 100. That is, the control unit 160 may control an operation of the ultrasonic probe 110, an operation of the signal processing unit 120, and an operation of the display unit 130. For example, by using a user command input through the user input unit 140 or a program stored in the storage unit 150, the control unit 160 may perform control such that the signal processing unit 120 generates an image. Also, the control unit 160 may perform control such that the image generated by the signal processing unit 120 is displayed on the display unit 130.

FIG. 2 is a block diagram of the ultrasonic probe 110 illustrated in FIG. 2. Referring to FIG. 2, the ultrasonic probe 110 may include a transmission unit 210, an ultrasonic transducer 220, and a reception unit 230.

The transmission unit 210 provides a driving signal to the ultrasonic transducer 220. The transmission unit 210 may include a pulse generating unit 212, a transmission delaying unit 214, and a pulser 216.

The pulse generating unit 212 generates a rate pulse for forming a transmission ultrasonic signal according to a predetermined pulse repetition frequency (PRF). The transmission delaying unit 214 applies a delay time for determination of transmission directionality to the rate pulse generated by the pulse generating unit 212. Rate pulses to which the delay time is applied correspond respectively to a plurality of ultrasonic cells 310 included in the ultrasonic transducer 220. The pulser 216 applies a driving signal (or a driving pulse) to the ultrasonic transducer 220 at a timing corresponding to each pulse to which the delay time is applied. The ultrasonic cells 310 may be a one-dimensional (1D) array type or a two-dimensional (2D) array type.

The ultrasonic transducer 220 transmits an ultrasonic signal to an object 10 according to the driving signal received from the transmission unit 210 and receives an echo signal reflected from the object 10. The ultrasonic transducer 220 may include the plurality of ultrasonic cells 310 that perform transduction between an ultrasonic signal and an electrical signal. The ultrasonic cells 310 may be a 1D array type or a 2D array type.

The ultrasonic transducer 220 may include a piezoelectric micromachined ultrasonic transducer (PMUT) that performs transduction between an ultrasonic signal and an electrical signal according pressure change, a capacitive micromachined ultrasonic transducer (CMUT) that performs transduction between an ultrasonic signal and an electrical signal according to an electrostatic capacity change, a magnetic micromachined ultrasonic transducer (MMUT) that performs transduction between an ultrasonic signal and an electrical signal according to a magnetic field change, and an optical ultrasonic detector that performs transduction between an ultrasonic signal and an electrical signal according to an optical characteristic change.

The reception unit 230 generates ultrasonic data by processing a signal received from the ultrasonic probe 220. The reception unit 230 may include an amplifier 232, an analog-digital converter (ADC) 234, a reception delaying unit 236, and a summing unit 238.

The amplifier 232 amplifies a signal received from the ultrasonic transducer 220, and the ADC 234 analog-digital converts the amplified signal. The reception delaying unit 236 applies a delay time for determination of reception directionality to the analog-digital converted signal. The summing unit 238 generates ultrasonic data by summing signals processed by the reception delaying unit 236. A reflection component in a direction determined by the reception directionality may be emphasized by a summing operation of the summing unit 238.

The ultrasonic probe 110 may necessarily include the ultrasonic transducer 220, but at least some elements of the transmission unit 210 and the reception unit 230 may be included in other devices. For example, the ultrasonic probe 110 may not include the summing unit 238 of the reception unit 230. FIG. 2 illustrates that the ultrasonic probe 110 includes one ultrasonic transducer 220; however, this is merely for convenience of description and the ultrasonic probe 110 may include a plurality of ultrasonic transducers 220. When the ultrasonic probe 110 includes a plurality of ultrasonic transducers 220, the transmission unit 210 and the reception unit 230 may be allocated to each of the ultrasonic transducers 220; however, embodiments are not limited thereto. A plurality of ultrasonic transducers 220 may be connected to one transmission unit 210 and one reception unit 230.

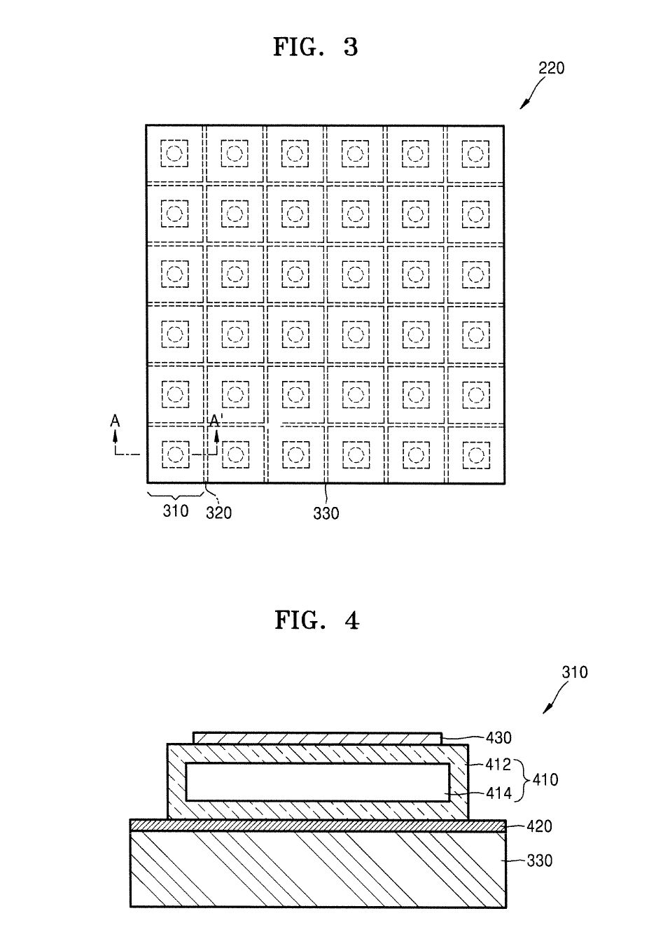

FIG. 3 is a plan view of the ultrasonic transducer 220 illustrated in FIG. 2, and FIG. 4 is a schematic cross-sectional view of an ultrasonic cell 310 illustrated in FIG. 3. As illustrated in FIG. 3, the ultrasonic transducer 220 may include a plurality of ultrasonic cells 310 and at least one current prevention portion 320 that prevents an electric current between the ultrasonic cells 310.

In the ultrasonic transducer 220, the ultrasonic cells 310 may be arranged in the form of an m×n array (m and n are natural numbers greater than or equal to 1). FIG. 3 illustrates that the ultrasonic cells 310 are arranged in the form of a 6×6 array; however, embodiments are not limited thereto. The current prevention portion 320 is provided between the ultrasonic cells 310 to prevent an electric current between the ultrasonic cells 310 so that the ultrasonic cells 310 are driven separately. The current prevention portion 320 may be formed of a hole passing through a substrate 330 included in the ultrasonic cell 310 and may not be electrically connected to a substrate 330 of the adjacent ultrasonic cell 310. Also, a bulk acoustic wave, which may be transmitted to the adjacent ultrasonic cell 310, may be blocked by the current prevention portion 320 to reduce interference between the ultrasonic cells 310.

FIG. 3 illustrates that the ultrasonic transducer 220 includes a plurality of ultrasonic cells 310 arranged two-dimensionally; however, embodiments are not limited thereto. The ultrasonic transducer 220 may include a plurality of ultrasonic cells 310 that are arranged one-dimensionally. When the ultrasonic transducer 220 includes a plurality of ultrasonic cells 310, the ultrasonic cells 310 of the ultrasonic transducer 220 operate at different frequencies. Thus, the ultrasonic transducer 220 may operate at multiband frequencies. An operation frequency of the ultrasonic cell 310 may be determined by at least one of the size, shape, and mechanical properties of the ultrasonic cell 310. By differently designing at least one of the size, shape, and mechanical properties of the ultrasonic cells 310, the ultrasonic cells 310 may operate at different frequencies.

As illustrated in FIG. 4, the ultrasonic cell 310 may include a vibration portion 410 that may vibrate according to an applied voltage, and first and second electrodes 420 and 430 that generate an electric field at the vibration portion 410. The ultrasonic cell 310 may be a CMUT cell.

The vibration portion 410 may include a membrane 412 and a cavity 414 formed in the membrane 412. The cavity 414 may be a capacitor and may be in a vacuum state. For example, the membrane 412 may be formed of monocrystalline silicon. A cross-section of the membrane 412 may be circular or polygonal; however, embodiments are not limited thereto. While at least a partial region of the membrane 412 is elastically deformed, the membrane 412 may vibrate in a direction perpendicular to the substrate 330. That is, the vibration portion 410 may vibrate vertically with respect to the substrate 330.

The first and second electrodes 420 and 430 may be spaced apart from each other and may contact a partial region of the membrane 412. The first and second electrodes 420 and 430 may be disposed symmetrically about a center of the ultrasonic cell 310; however, embodiments are not limited thereto. The first and second electrodes 420 and 430 may be formed of a conductive material, for example, such as cuprum (Cu), aluminum (Al), aurum (Au), chromium (Cr), molybdenum (Mo), titanium (Ti), or platinum (Pt). One of the first and second electrodes 420 and 430 may be formed in common in the ultrasonic cells 310. For example, the first electrode 420 may be formed in common in the ultrasonic cells 310, and the second electrode 430 may be formed in each of the ultrasonic cells 310.

The first electrode 420 may receive an input of a voltage from an external ground or a direct current (DC) bias signal source. Thus, an electric charge may be prevented from being stored in the membrane 412. Accordingly, the ultrasonic transducer 220 may operate stably without a characteristic change with time. Also, the second electrode 430 may apply an electrical signal, that is, a current, from an external signal source, and may transfer an electrical signal change, for example, an electrostatic capacity change of the cavity 414 in the membrane 412 to an outside thereof.

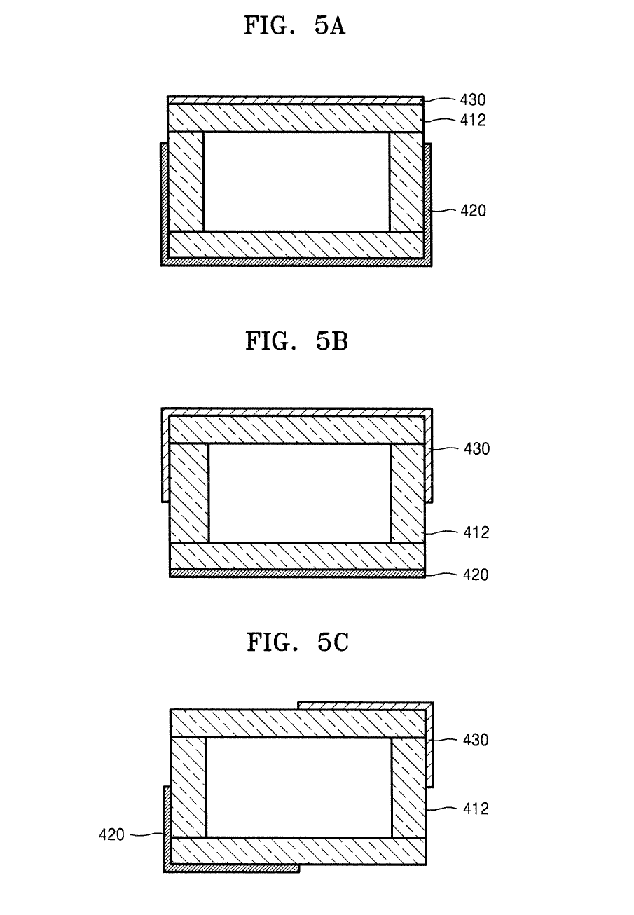

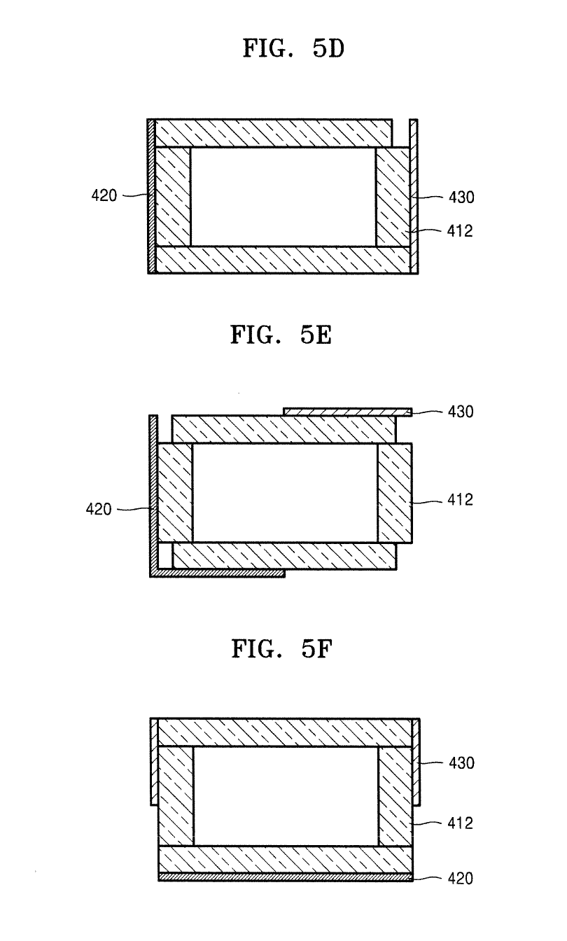

FIGS. 5A to 5F are cross-sectional views illustrating various examples of an electrode arrangement of the ultrasonic cell 310.

The first and second electrodes 420 and 430 disposed in the ultrasonic cell 310 may be spaced apart from each other and may contact a partial region of the membrane 412. For example, as illustrated in FIG. 5A, the first electrode 420 may be disposed in a partial region of bottom and side surfaces of the membrane 412, and the second electrode 430 may be disposed on a top surface of the membrane 412. Also, as illustrated in FIG. 5B, the first electrode 420 may be disposed on a bottom surface of the membrane 412, and the second electrode 430 may be disposed in a partial region of top and side surfaces of the membrane 412. Also, as illustrated in FIG. 5C, the first electrode 420 may be disposed in a partial region of bottom and side surfaces of the membrane 412, and the second electrode 430 may be disposed in a partial region of top and side surfaces of the membrane 412. Also, as illustrated in FIG. 5D, the first electrode 420 may be disposed on a side surface of the membrane 412, and the second electrode 430 may be disposed on an opposite side surface of the membrane 412. Also, as illustrated in FIG. 5E, the first electrode 420 may be disposed in a partial region of bottom and side surfaces of the membrane 412, and the second electrode 430 may be disposed in a partial region of a top surface of the membrane 412. Also, as illustrated in FIG. 5F, the first electrode 420 may be disposed on a bottom surface of the membrane 412, and the second electrode 430 may be disposed on side surfaces of the membrane 412.

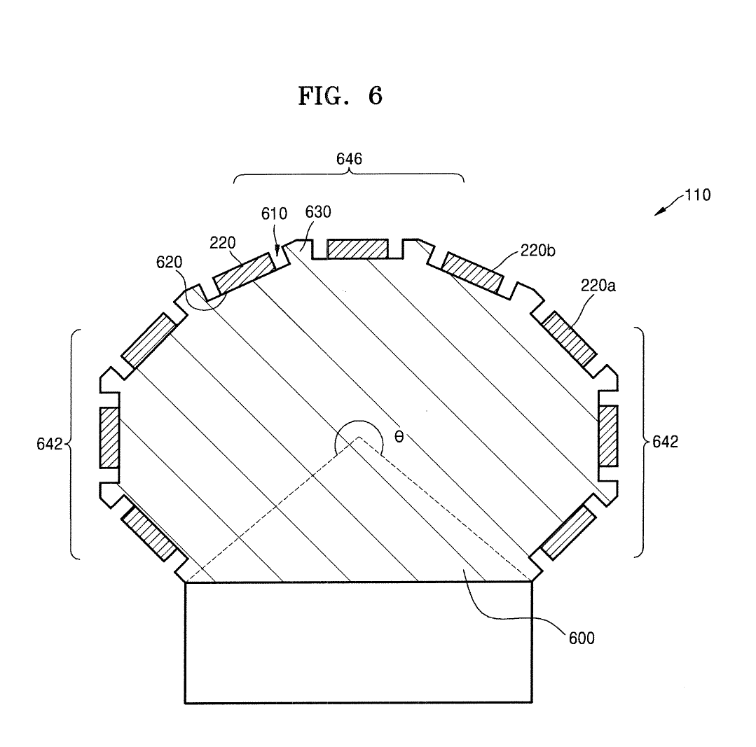

The ultrasonic transducer 220 may be disposed in a groove 610 in a body 600. FIG. 6 is a schematic cross-sectional view of the body 600 in which the ultrasonic transducers 220 are arranged according to an embodiment of the present invention. As illustrated in FIG. 6, the body 600 may form an external appearance of the ultrasonic probe 110, and a plurality of grooves 610 may be formed in the body 600. The ultrasonic transducers 220 may be disposed respectively in the grooves 610 and may perform transduction between an ultrasonic signal and an electrical signal. For example, the ultrasonic transducer 220 may be disposed on a bottom surface 620 of the groove 610. The bottom surface 620 may be flat. Thus, an ultrasonic signal generated by the ultrasonic transducer 220 may be transmitted to an outside of the body 600 in a direction perpendicular to the bottom surface 620. A cross-section of the groove 610 may be circular, elliptical, or polygonal.

The body 600 may include a curved surface, and the grooves 610 may be formed on the curved surface. For example, the curved surface of the body 600 may be convexed outward. Thus, the ultrasonic probe 110 may have an ultrasonic transmission angle θ of about 120° to about 360°. When the ultrasonic transducer 220 is a CMUT, it may be formed by stacking a vibration portion and electrodes. Thus, the ultrasonic probe 110 including a CMUT has a small ultrasonic transmission angle θ. However, as in an embodiment, when the ultrasonic probe 110 is manufactured by forming the grooves 610 in the body 600 having a curved surface and disposing the ultrasonic transducers 220 in the grooves 610, the ultrasonic transmission angle θ of the ultrasonic probe 110 is not restricted even when the ultrasonic probe 110 is a CMUT. Thus, the ultrasonic probe 10 according to an embodiment may also be applied to a small-sized probe such as an endocavity or endovarginal heart 3D image diagnosis probe.

In order to increase the ultrasonic transmission angle θ, the grooves 610 are formed in the curved surface of the body 600; however, this is merely an example. The grooves 610 may also be formed in a flat surface of the body 600. The ultrasonic transducers 220 may be arranged in the form of a phased array, a linear array, or a convexed array.

The body 600 may be formed of a sound-absorbing material. The body 600 may support the ultrasonic transducer 220 and may absorb an ultrasonic signal that is transmitted to a backside of the ultrasonic transducer 220 and is not used in a test or diagnosis. Although other elements of the ultrasonic probe 110, except for the ultrasonic transducer 220, are not illustrated, they may be provided in the body 600.

The grooves 610 may be arranged one-dimensionally or two-dimensionally. For example, the grooves 610 may be spaced apart from each other. The ultrasonic transducers 220 may be disposed respectively in the grooves 610. A spacing portion 630, which is a portion of the body 600, may be disposed between the grooves 610. The spacing portion 620 may be formed of a sound-absorbing material that is identical to the material of the body 600. Thus, crosstalk between the adjacent ultrasonic transducers 220 may be reduced.

A frequency of an ultrasonic signal transmitted by a first ultrasonic transducer 220a may be different from a frequency of an ultrasonic signal transmitted by a second ultrasonic transducer 220b. For example, the first ultrasonic transducer 220a operating at a first frequency may be disposed in the groove 610 in a first region 642 of the body 600, and the second ultrasonic transducer 220b operating at a second frequency may be disposed in the groove 610 in a second region 646 of the body 600. Since the ultrasonic transducers 220 operating at different frequencies are disposed in one body 600, the ultrasonic probe 110 may operate at multiband frequencies. Thus, a diagnosis or treatment may be performed by one ultrasonic probe, regardless of the types of objects.

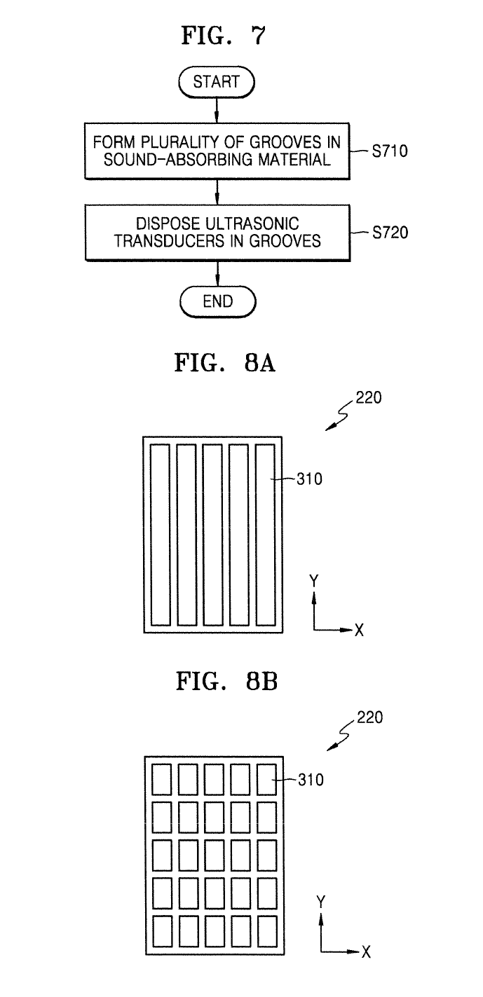

FIG. 7 is a flowchart of a method of manufacturing an ultrasonic probe, according to an embodiment of the present invention. First, a plurality of grooves 610 are formed in a sound-absorbing material (S710). The sound-absorbing material as a body 600 is formed by using a mold or the like. The body 600 may have a curved surface. The grooves 610 are formed in the curved surface of the body 600. A bottom surface 620 of the groove 610 may be flat. The grooves 610 may be spaced apart from each other and may be arranged one-dimensionally or two-dimensionally. A spacing portion 630, which is a portion of the body 600, may be formed between the grooves 610.

Thereafter, ultrasonic transducers 220 performing transduction between an ultrasonic signal and an electrical signal are disposed respectively in the grooves 610 (S720). At least two of the ultrasonic transducers 220 may transmit ultrasonic signals of different frequencies. Thus, the ultrasonic probe according to an embodiment of the present invention may operate at multiband frequencies. As described above, since the ultrasonic probe is manufactured by attaching a plurality of ultrasonic transducers 220 to the body 600, durability may be improved as compared to the case where a plurality of ultrasonic transducers 220 are divided by a dicing process.

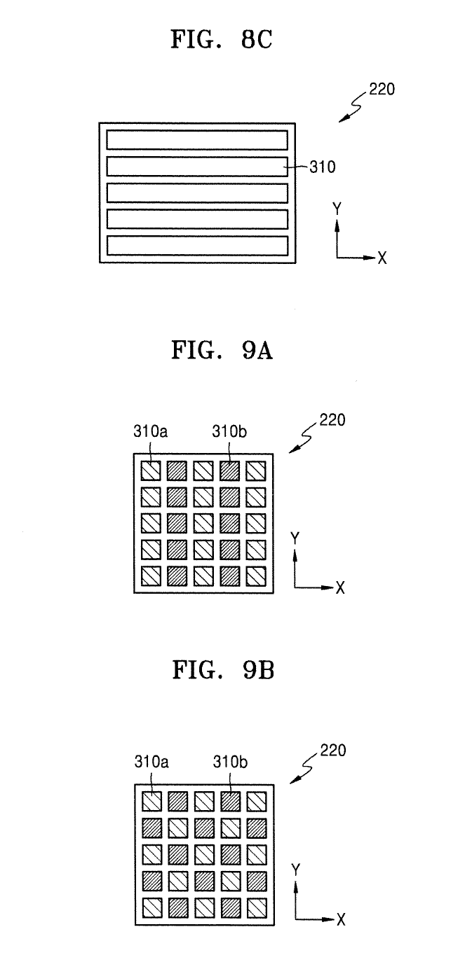

As described above, when the ultrasonic transducer 220 includes a plurality of ultrasonic cells 310, the plurality of ultrasonic cells 310 may be arranged one-dimensionally or two-dimensionally. FIGS. 8A to 8C illustrate ultrasonic transducers according to embodiments of the present invention. As illustrated in FIG. 8A, each of the ultrasonic cells 310 may be elongated in the Y-axis direction and the ultrasonic cells 310 may be arranged in the X-axis direction. As illustrated in FIG. 8B, the ultrasonic cells 310 may be arranged two-dimensionally. As illustrated in FIG. 8C, each of the ultrasonic cells 310 may be elongated in the X-axis direction and the ultrasonic cells 310 may be arranged in the Y-axis direction.

As described above, the ultrasonic transducer 220 includes a plurality of ultrasonic cells operating at different frequencies. FIGS. 9A and 9B illustrate an arrangement relation between the ultrasonic cells operating at different frequencies, according to an embodiment of the present invention. As illustrated in FIG. 9A, the ultrasonic transducer 220 may include a first ultrasonic cell 310a operating at a first frequency and a second ultrasonic cell 310b operating at a second frequency. The first ultrasonic cell 310a and the second ultrasonic cell 310b may be disposed alternately in a first direction (e.g., the X-axis direction). However, ultrasonic cells operating at the same frequency may be arranged in a second direction (e.g., the Y-axis direction) intersecting the first direction. That is, the first ultrasonic cell 310a or the second ultrasonic cell 310b may be arranged one-dimensionally in the second direction.

As illustrated in FIG. 9B, the ultrasonic transducer may include a first ultrasonic cell 310a operating at a first frequency and a second ultrasonic cell 310b operating at a second frequency. The first ultrasonic cell 310a and the second ultrasonic cell 310b may be arranged alternately in the first direction (e.g., the X-axis direction) and the second direction (e.g., the Y-axis direction). That is, the second ultrasonic cell 310b may be disposed in a region that is adjacent to the first ultrasonic cell 310a in the first direction and the second direction.

FIGS. 9A and 9B illustrate two different ultrasonic cells 310a and 310b that are arranged in one ultrasonic transducer. However, embodiments of the present invention are not limited thereto, and three or more ultrasonic cells may be arranged in one ultrasonic transducer.

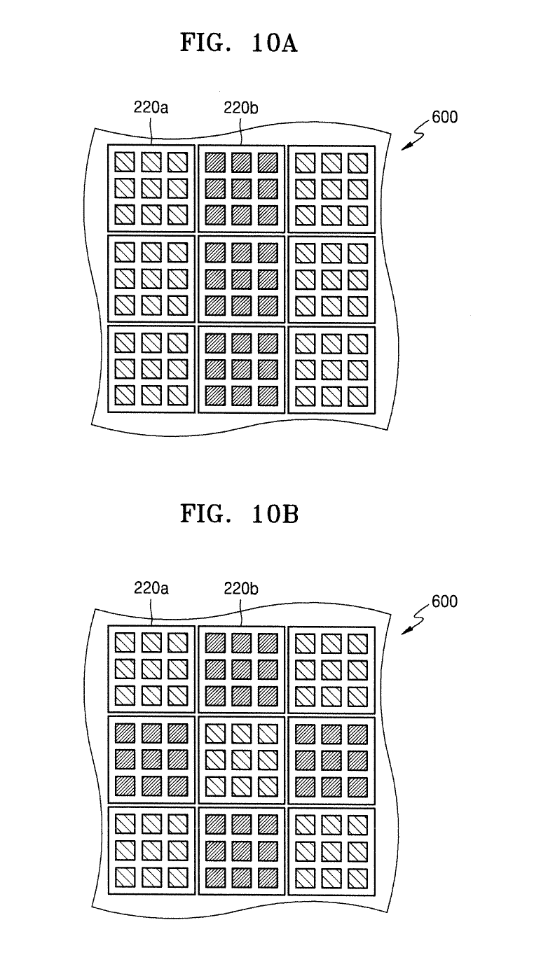

As described above, the ultrasonic probe includes a plurality of ultrasonic transducers, and the plurality of ultrasonic transducers includes at least two ultrasonic cells operating at different frequencies. FIGS. 10A and 10B illustrate an arrangement relation between the ultrasonic transducers operating at different frequencies, according to an embodiment of the present invention. FIGS. 10A and 10B are partial plan views of the body of the ultrasonic probe.

As illustrated in FIG. 10A, the ultrasonic probe may include a first ultrasonic transducer 220a operating at a first frequency and a second ultrasonic transducer 220b operating at a second frequency. The first ultrasonic transducer 220a and the second ultrasonic transducer 220b may be disposed alternately in a first direction (e.g., the X-axis direction). However, ultrasonic transducers operating at the same frequency may be arranged in a second direction (e.g., the Y-axis direction) intersecting the first direction. That is, the first ultrasonic transducer 220a or the second ultrasonic transducer 220b may be arranged one-dimensionally in the second direction.

As illustrated in FIG. 10B, the ultrasonic probe may include a first ultrasonic transducer 220a operating at a first frequency and a second ultrasonic transducer 220b operating at a second frequency. The first ultrasonic transducer 220a and the second ultrasonic transducer 220b may be arranged alternately in the first direction (e.g., the X-axis direction) and the second direction (e.g., the Y-axis direction). That is, the second ultrasonic transducer 220b may be disposed in a region that is adjacent to the first ultrasonic transducer 220a in the first direction and the second direction.

FIGS. 10A and 10B illustrate one ultrasonic transducer that includes a plurality of ultrasonic cells operating at the same frequency. However, embodiments of the present invention are not limited thereto, and one ultrasonic transducer may include a plurality of ultrasonic cells operating at different frequencies.

It should be understood that the exemplary embodiments described herein should be considered in a descriptive sense only and not for purposes of limitation. Descriptions of features or aspects within each embodiment should typically be considered as available for other similar features or aspects in other embodiments.

While one or more embodiments of the present invention have been described with reference to the figures, it will be understood by those of ordinary skill in the art that various changes in form and details may be made therein without departing from the spirit and scope of the present invention as defined by the following claims.

What is claimed is:

1. An ultrasonic probe comprising:
a body in which a plurality of grooves are formed; and
a plurality of ultrasonic transducers disposed respectively in the grooves to perform transduction between an ultrasonic signal and an electrical signal.

2. The ultrasonic probe of claim 1, wherein the body comprises a sound-absorbing material.

3. The ultrasonic probe of claim 1, wherein the ultrasonic transducers are disposed on bottom surfaces of the grooves.

4. The ultrasonic probe of claim 3, wherein the bottom surfaces are flat.

5. The ultrasonic probe of claim 3, wherein the ultrasonic transducers transmit ultrasonic signals in directions perpendicular to the bottom surfaces of the grooves.

6. The ultrasonic probe of claim 1, wherein
the grooves comprise a first groove and a second groove that are spaced apart from each other, and
the ultrasonic transducers comprise a first ultrasonic transducer and a second ultrasonic transducer that are disposed respectively in the first groove and the second groove.

7. The ultrasonic probe of claim 6, wherein a spacing portion, which is a portion of the body, is disposed between the first groove and the second groove.

8. The ultrasonic probe of claim 6, wherein a frequency of an ultrasonic signal transmitted by the first ultrasonic transducer is different from a frequency of an ultrasonic signal transmitted by the second ultrasonic transducer.

9. The ultrasonic probe of claim 1, wherein
the body comprises a curved surface, and
the grooves are formed in the curved surface.

10. The ultrasonic probe of claim 9, wherein the curved surface is convexed outward.

11. The ultrasonic probe of claim 1, wherein the ultrasonic probe has an ultrasonic transmission angle of about 120° to about 360°.

12. The ultrasonic probe of claim 1, wherein the grooves are arranged one-dimensionally or two-dimensionally.

13. The ultrasonic probe of claim 1, wherein the ultrasonic transducers are capacitive micromachined ultrasonic transducers.

14. The ultrasonic probe of claim 13, wherein the ultrasonic transducers comprise at least one ultrasonic cell comprising a vibration portion vibrating according to an applied voltage, a first electrode contacting the vibration portion, and a second electrode contacting the vibration portion and spaced apart from the first electrode.

15. The ultrasonic probe of claim 12, wherein the ultrasonic transducers comprise a first ultrasonic cell vibrating in a first frequency band and a second ultrasonic cell vibrating in a second frequency band.

16. A method of manufacturing an ultrasonic probe, the method comprising:
forming grooves in a sound-absorbing material of a body; and
disposing ultrasonic transducers, which perform transduction between an ultrasonic signal and an electrical signal, respectively in the grooves.

17. The method of claim 16, wherein a spacing portion, which is a portion of the body, is formed between the grooves.

18. The method of claim 16, wherein the grooves are formed in a curved surface of the body.

19. The method of claim 16, wherein the grooves have flat bottom surfaces.

20. The method of claim 16, wherein the ultrasonic probe operates at multiband frequencies.
